(19) United States
(12) Patent Application Publication
(10) Pub. No.: US 2009/0270803 A1
(43) Pub. Date: Oct. 29, 2009

(54) SAFETY DEVICE FOR INJECTING A MEDICINAL PRODUCT, SUCH AS A LYOPHILIZED PRODUCT

(76) Inventor: Marc Brunel, Toulouse (FR)

Correspondence Address: BIRCH STEWART KOLASCH & BIRCH, PO BOX 747, FALLS CHURCH, VA, 22040-0747, US

(21) Appl. No.: 12/304,705

(22) PCT Filed: Jun. 13, 2007
(86) PCT No.: PCT/FR2007/000979

§ 371 Date: Dec. 12, 2008

Related U.S. Application Data
(60) Provisional application No. 60/814,071, filed on Jun. 16, 2006.

(51) Int. Cl. A61M 5/50

(52) U.S. Cl. 604/110

(57) ABSTRACT

The invention relates to a safety device for injecting a medicinal product, such as a lyophilized product, comprising a syringe body (9), a protective casing (19) and locking members (22, 23) for maintaining the syringe body (9) and the protective casing (19) in an injection position. According to the invention, this device comprises members (40) which immobilize the locking members (22, 23) and which can be displaced between a natural initial position, called the deactivation position, in which each locking member (22, 23) is immobilized in a locked state, and a position, called the activation position, which allows each locking member (22, 23) to be switched to an unlocked state. Moreover, actuating members (46), positioned on the trajectory of an intermediate shoulder (32) formed on the plunger rod (6) of the injection device, are designed to bring about a displacement of the blocking members from their deactivation position to their activation position during the ascending movement of the plunger rod.

The invention relates to a safety device for injecting a medicinal product such as a lyophilized product.

For obvious safety reasons, current single use injection devices are commonly equipped with a protection device after using the injection needle.

Normally, such injection devices comprise a syringe body comprising a receptacle with an injection needle and equipped with a plunger rod equipped with a push-button, and a protection device comprising:
a protective casing adapted so that said protective casing and the syringe body are able to slide one in relation to the other between an injection position in which the injection needle extends at least partially into the extension of the protective casing and a protection position after use in which the injection needle is completely accommodated in the protective casing,
and at least one locking member which can have a locked state of holding the syringe body and the protective casing in their injection position, and to switch to an unlocked state able to allow, at the end of the movement of the rod inside the receptacle, the relative sliding of the syringe body and the protective casing towards their protection position after use.

It is also to be noted that this principle applies both to devices equipped with an injection needle sealed on the receptacle and injection devices comprising an injection needle added and joined on in a detachable way by any assembly system known per se (Luër cone . . . ).

The design itself of these injection devices (activating the protection at the end of the movement of the rod) however prevents them from being used in particular for the injection of medicaments requiring a reconstitution (solvent-lyophilized product) before injection, and requiring, for this:
a first emptying for injecting the solvent contained in the injection device into a bottle containing the lyophilized product,
drawing up the reconstituted product in order to fill the injection device
and the injection itself of the medicinal product.

Indeed, their design would lead to the activation of the protection after the first emptying preventing the two subsequent steps.

In order to overcome this defect, a solution in particular described in the patent applications FR 2861310 et WO 02/072182 consisted in equipping the injection devices with an “inhibitory member” adapted:
due to its presence only, or in a first position of the latter, for shortening the movement of the rod during the first emptying, in order to avoid the activation of protection at the end of movement,
after its withdrawal, or in a second position of the latter, for allowing, during the injection itself, a normal movement of the rod causing the activation of the protection at the end of the movement.

Such a solution has the advantage of only requiring the addition of a simple “inhibitory member” in order to achieve the expected result.

Whereas, it turns out that such a solution that required a specific action deviating from the natural motions used for injecting reconstituted products, has been poorly accepted by medical staff, so that the devices designed according to this principle have in fact had very little commercial success.

The present invention aims to overcome this disadvantage and has the main objective of providing a safety device for injection allowing the injection of products to be reconstituted according to a methodology strictly identical to that used during the use of non-safety injection devices.

Another objective of the invention is to provide a safety device for injection resulting in a very slight additional cost compared to current non-safety injection devices.

To this end, the invention applies to a device for injecting a medicinal product such as a lyophilised product with the specificity of including a protection device comprising:
a shoulder placed in an intermediate position of the length of the plunger rod,
a locking member attached on one of the members, syringe body or protective casing, comprising:
   blocking devices of the locking members, which can be displaced between a natural initial position, called the deactivation position, of blocking each locking member in its locked state, and a position, called the activation position, which allows each locking member to be switched to its unlocked state,
and actuating members connected to the blocking devices, positioned on the trajectory of the intermediate shoulder of the plunger rod, and designed to undergo a change of state bringing about a displacement of said blocking devices from their deactivation position to their activation position when a force is exerted on said actuating members by the intermediate shoulder, during a displacement of the plunger rod resulting from a tension exerted on the push-button.

Therefore, in principal, the invention needs, in order to fulfil the objective cited, to equip the traditional safety injection devices with a locking element and to attach the latter, for example by detent, on one of the members, syringe body or protective casing, of these injection devices. As a result, the additional cost generated by the solution according to the invention proves very slight.

In addition, the transition of the blocking devices from their natural deactivation position of the locking members and their activation position of these locking members, operates automatically, without human intervention, when the reconstituted product is drawn up to fill the injection device.

As a result, the methodology used for the injection of products to be reconstituted proves strictly identical to that used during the use of non-safety injection devices.

According to a first advantageous variant of the embodiment of the invention:
the locking element is attached on one of the members, syringe body or protective casing, with the ability of displacement according to a direction parallel to the longitudinal axis of the protection device, between two positions defining the respective positions of deactivation and activation of the blocking devices of the locking members,
a contact area situated on the trajectory of the intermediate shoulder of the plunger rod,
and at least one buttressing part which can cause a displacement of the locking member resulting in a displacement of the blocking devices from their deactivation position to their activation position, at the time of a rotation of the lever brought about by the displacement of the plunger rod resulting from tension exerted on the push-button.

According to a second advantageous embodiment of the invention:
the locking member is attached in a fixed position on one of the members, syringe body or protective casing,
the actuating members comprise at least one locking part interdependent on the locking member, comprising a single holder
   contact area situated on the trajectory of the intermediate shoulder of the plunger rod,
   at least one blocking device of a locking member,
   and a deformable connection area between the contact area of each blocking device, which can be deformed and bring about a displacement of each blocking device from its deactivation position to its activation position, when a force is exerted on the contact area by the intermediate shoulder, during a displacement of the plunger rod resulting from a tension exerted on the push-button.

Furthermore, according to the invention, the locking member is advantageously attached on the syringe body.

Also, in order to perfect the locking of the locking fingers, the blocking devices of the locking member and said locking members advantageously include additional detachable assembly members, of the catch/pin type.

Furthermore, when the locking members, for maintaining the syringe body and the protective casing in their injection position, consist in the normal way of flexible strips, the blocking devices of said locking members are then advantageously adapted, according to the invention, to prevent the latter's deformation by bending, in their deactivation position.

Other features, purposes and advantages of the invention will emerge from the detailed description that follows with reference to the annexed drawings that represent by way of non-limiting examples two preferred methods of embodiment of the invention. In these drawings:

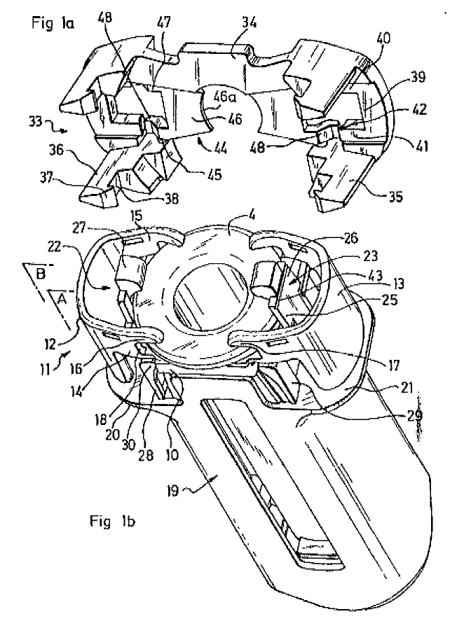

FIG. 1a is a perspective view representing a locking member adapted to equip a first method of embodiment of an injection device according to the invention represented seen in perspective in FIG. 1b,

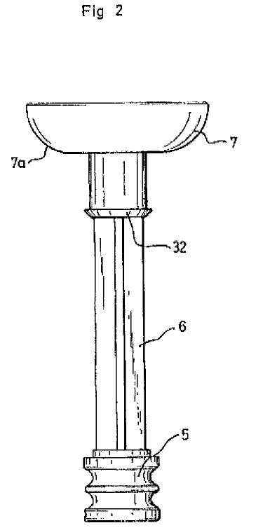

FIG. 2 is a longitudinal view of a plunger rod of an injection device according to the invention,

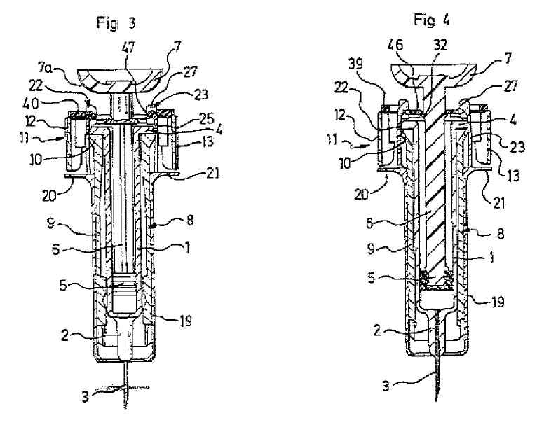

FIGS. 3 and 4 are longitudinal sections respectively on the plane A and axial plan B, of the first method of embodiment of the injection device in accordance with the invention represented in perspective in FIGS. 1a and 1b,

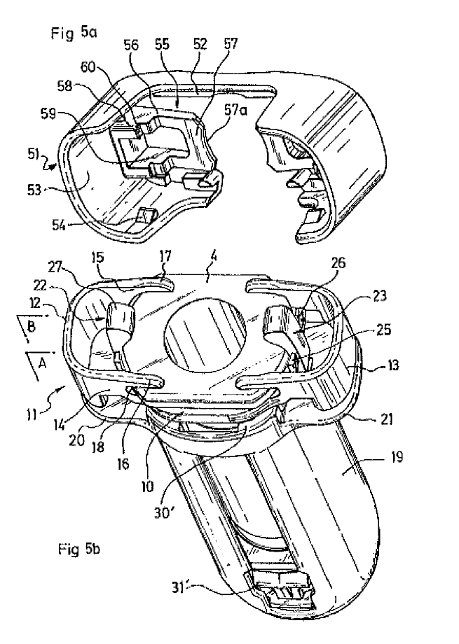

FIG. 5a is a perspective view representing a locking element adapted for equipping a second method of embodiment of an injection device according to the invention represent in perspective view in FIG. 5b.

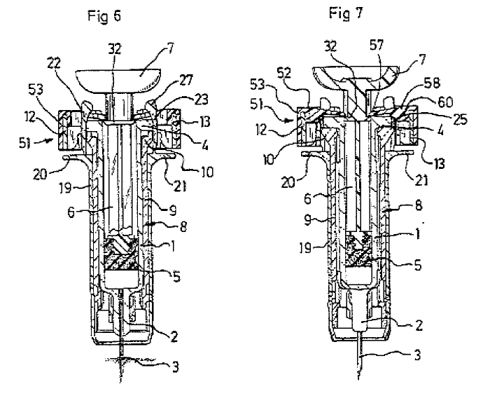

FIGS. 6 and 7 are longitudinal sections respectively on the axial plane B and the plane A, of the second method of embodiment of the injection device in accordance with the invention represented in perspective in FIGS. 5a and 5b,

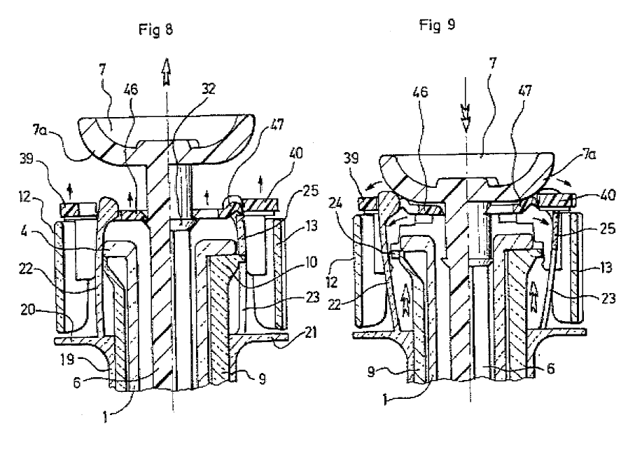

FIGS. 8 and 9 are two partial longitudinal sections of the first method of embodiment of the injection device in accordance with the invention each representing, in half section on the plane A and in half section on the plane B, one of the steps of operation of this injection device,

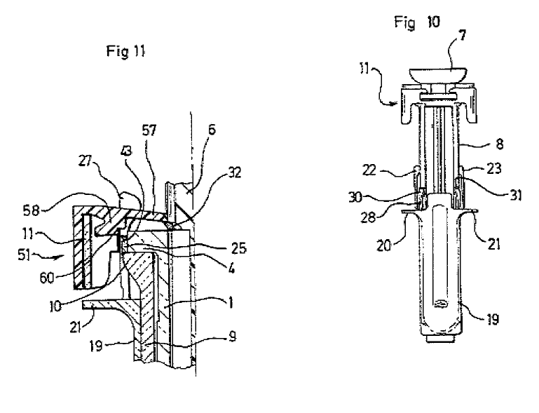

FIG. 10 is a longitudinal view of the first method of embodiment of the injection device in accordance with the invention representing the latter during the final step of the end of injection,

and FIG. 11 is a partial longitudinal section of the second method of embodiment of the injection device in accordance with the invention representing, in half section on the plane A and in half section on the plane B, the step of operation of this injection device corresponding to that represented in FIG. 8 with reference to the first injection device.

The two variants of injection devices according to the invention represented by way of examples in the figures are of the single use type and consist of injection devices especially for injection of medicaments requiring a reconstitution (solvent-lyophilized product) before injection, and requiring, for this:
a first emptying destined to inject the solvent contained in the injection device into a bottle containing the lyophilized product,
a drawing up of the reconstituted product in order to fill the injection device,
and the injection itself of the medicinal product.

In addition, these two variants of injection devices integrate protection devices adapted to enable their injection needle to be protected after use.

Firstly, each of the injection devices comprises a receptacle such as in the example, a traditional syringe 1 constituted of a glass tube, on one hand, extended by a base part 2 into which an injection needle 3 is sealed, and on the other hand, equipped with an external collar 4 on its end opposite the base part 2.

In addition, this syringe is traditionally equipped with a plunger 5 able to slide in a sealed way inside the latter, and attached, in order to actuate it, on one end of a plunger rod 6 equipped with a push-button 7 at its opposite end that has the shape of a bowl, i.e. which has a face 7a joining with the plunger rod 6, of convex shape.

As stated above, these two injection devices also comprise a device for protecting the injection needle 3 after use. According to the two examples represented in the figures, these two protection devices consist of devices of the same basic design as that of the injection devices commercialized by the company “SAFETY SYRINGES, Inc.”, and described in particular in patent applications WO 2006/127484 and WO 2006/050304, patents U.S. Pat. No. 6,030,366 and U.S. Pat. No. 6,623,459, these documents being considered, due to their referencing, as being an integral part of the present description.

Firstly, the protection device of the first injection device represented in FIGS. 1b, 3 and 4, has a syringe body 8 of the type described in the patent application WO 2006/050304, comprising a longitudinal shaft 9 defining a slot for the syringe 1, equipped, at one of its ends, with an external restrainer and support flange 10 of the collar 4 of said syringe.

This syringe body 8 also has a finger grip tubular head 11, placed around the end section of the longitudinal shaft 9 on which the flange 10 is formed.

This tubular head 11 is formed of two half shells 12, 13 which extend symmetrically on both sides of the flange and each connected to the latter by two parallel arms, each, extended by an elastically deformable trigger 16, 17, and adapted so that the triggers 16, 17 of the two coplanar arms define, with the face facing the flange 10, an opening 18 for accommodating and blocking the external collar 4 of the syringe 1.

The protection device of the first injection device in accordance with the invention also comprises a protective casing 19 adapted to slide externally along the tubular shaft 9 of the syringe body 8.

This protective casing 19 has, firstly, two finger grip shields 21, 22 arranged and of shapes adapted to approximately block off the open face in relation to the tubular head 11.

In addition, this protective casing 19 has two locking fingers 22, 23 arranged to symmetrically extend on both side of the tubular shaft 9, each from one of the finger grip shields 20, 21, and adapted to block the protective casing 19 in translation relative to the syringe body 8, in a relative retracted position of said protective casing where the injection needle 3 extends beyond the latter.

Each one of these locking fingers 22, 23 is composed of an elastically deformable strip extending beyond the collar 4 of the syringe 1 blocked in the openings 18, and extending in particular, for this purpose inside a slot 24, shown in FIG. 9, arranged on the periphery of the flange 10 of the syringe body 8.

Each strip 22, 23 also comprises an intermediate locking member adapted to come to rest and lock on the flange 10 on both sides of the slot 24 arranged in the latter, said locking member consisting of a cross member defining two locking tabs 25, 26 extending on both sides of said strip.

Each strip 22, 23 has, lastly, a guard 27 arranged on its free end and adapted to form an additional support face of the convex face of the push button 7 of the plunger rod 6, which can activate an unlocking of the locking tabs 25, 26 by bending each strip 22, 23 at the end of movement of said plunger rod 6.

The protective casing also has four locking fingers like 28, 29, distributed around the tubular shaft 9, arranged for each to extend from one of the finger grip shields 20, 21, and adapted to block the protective casing 19 in translation relative to the syringe body 8, in a relative opened out position of said protective casing where the injection needle 3 is completely accommodated in the latter.

Each of these locking fingers 28, 29 is composed of an elastically deformable strip, equipped, at its free end, with a locking pin 30 adapted to come and lodge itself in a catch 31 arranged along the longitudinal shaft 9, and shown in FIG. 10.

Normally, the protection device also has a spring (not shown) arranged between the syringe body 8 and the protective casing 19 and adapted to bring about a relative sliding of the latter at the end of injection.

According to the invention and, firstly, the plunger rod 6 has a collar 32 arranged in an intermediate position of the length of said plunger rod.

According to the invention, furthermore, the protection devices have a locking member adapted to collaborate with the above mentioned collar 32 in order to automatically have, a state which can:
prevent the activation of unlocking the unlocking tabs 25, 26, during the first actuation of the plunger rod 6 intended to enable the mixing of the product,
and allow the activation of unlocking the unlocking tabs 25, 26, during the second actuation of the plunger rod intended for the injection of the product.

In regard to the protection device of the first method of embodiment of the injection device according to the invention, this locking member 33 is adapted to fit into the inside of the head of the finger grip 11.

It comprises a locking plate 34 adapted to extend approximately in the plane of the face of the head of the finger grip opposite the finger grip shields 20, 21, in relation to which tour feet extend like 35, 36 adapted to extend inside the head of the finger grip 11, and ending in a hook 37 delimiting a longitudinal groove 38 adapted to accommodate, with displacement play, the edge of the flange 10.

The locking plate 34 comprises, furthermore, in regard to each locking finger 22, 23, a U shaped cut out 39 defining two parallel branches 40, 41 adapted for each to be positioned in abutment against a locking tab 25, 26.

In order to perfect the locking of the locking fingers 22, 23, the end face of each branch 40, 41 has, furthermore, the shape of a pin 42 adapted to fit into a catch 43 of complementary shape arranged in the face facing each locking tab 25, 26.

This locking plate 34 also comprises, associated with each locking finger 22, 23, a lever 44 articulated around a rotation axis 45 orthogonal to the longitudinal axis of the syringe body 8, said lever comprising:
flat abutment part 46 of the intermediate collar 32 of the plunger rod 6 extending on one of the sides of the rotation axis 45, arranged so as to be situated on the trajectory of said collar, said abutment part also having a free bevelled edge 46a which can facilitate the passage of the collar 32 during a displacement of the plunger 5 in the direction of emptying the syringe 1,
and, extending on the other side of the rotation axis 45, two parallel branches 47, 48 each adapted to come to rest, at its free end, on a locking tab 25, 26.

The operation of this first injection device is described below with reference to FIGS. 3, 4, 8, 9 and 10.

In the initial position of the injection device represented in FIGS. 3 and 4, the locking member 33 is positioned so that the pins 42 of the branches 40, 41 are accommodated in the catches 43 of the locking tabs 25, 26.

This locking member 33 is thus in a position called the deactivation position in which any relative moving apart of the locking fingers 22, 23, is prevented, and therefore any unlocking of said locking fingers.

The first actuation of the plunger 5 with the purpose of emptying the syringe in order to mix the solvent and the lyophilized product, does not cause any change of the position of the locking member 33: the passing by the intermediate collar 32 of the plunger rod 6 of the obstacle that constitutes the abutment part 46 only causes, in effect, a tilting of no consequence of the lever 44.

Whereas, as represented in FIG. 8, during the drawing up of the mixture inside the syringe 1, the intermediate collar 32 comes up against the abutment part 46 and brings about a tilting of the lever 44 which causes a buttressing of the branches 47, 48 on the locking tabs 25, 26: the locking member 33 undergoes an axial movement and comes into a position in a state called the activation position in which the branches 40, 41 are arranged beyond the locking tabs 25, 26 and therefore no longer prevent the relative moving apart of the locking fingers 22, 23.

Due to this, and as represented in FIG. 9, when the plunger 5 reaches the end of the movement, the push-button 7 activates the unlocking of the unlocking fingers 22, 23, and thus brings about a relative sliding of the syringe body 8 and of the protective casing 19 until a relative opened out position of the protective casing 19 is obtained, represented in FIG. 10, carried out by the cooperation of the locking pins 30 and the catches 31, and in which the injection needle 3 is accommodated inside said protective casing.

The protection device of the second method of embodiment of the injection device according to the invention represented in FIGS. 5b, 6 and 7, has a syringe body 8 and a protective casing similar to those described above.

The only significant differences are relative to the locking members adapted to block the protective casing 19 in translation relative to the syringe body 8 in the relative opened out position of said protective casing where the injection needle 3 is completely accommodated in the latter.

According to this method of embodiment, in effect these locking members comprise:
an opening 30′ arranged in the peripheral wall of the head of the finger grip 11,
and a catch 31′ formed on the longitudinal shaft 9 of the syringe body 8, towards the free end of the latter.

Likewise, the plunger rod 6 is similar to that described above and represented in FIG. 2.

The essential difference between these two methods of embodiment concerns the locking member.

According to the second method of embodiment, the locking member 51 is adapted to cover the head of the finger grip 11.

It comprises a locking plate 52 adapted to extend approximately in the plane of the face of the head of the finger grip 11 set against the shields of the finger grip 20, 21, in relation to which a skirt 53 extends adapted to cover the two half shells 12, 13.

In addition, this locking member 51 comprises pins like 54 arranged projecting in relation to the internal face of the skirt 53 and adapted to hook without play under the arms 14, 15 of the tubular head 11.

This locking member 51 also comprises, associated with each locking finger 22, 23, a locking part 55 arranged inside the skirt 53, said locking part having an appropriate elasticity and being connected to the skirt 53 by a connection such that it has an ability of elastic deformation.

This locking part 55 has:
a frame 56 defining an opening for the passage of one of the locking fingers 22, 23, said frame also having an abutment cross-piece 57 of the intermediate collar 32 of the plunger rod 6 arranged so as to be situated on the trajectory of said collar, said abutment cross-piece also having, a free bevelled edge 57a for facilitating the passage of the collar 32 during a displacement of the plunger 5 in the direction of emptying the syringe 1,
two branches 58, 59 each one extending under one of the uprights of the frame 56 perpendicular to the cross-piece 57, so as to position each one in abutment against a locking tab 25, 26 of the locking fingers 22, 23, said branches having an end face forming a pin adapted to be accommodated in the catch 43 of the locking tab 25, 26 opposite.

According to this principle the locking member 51 is attached with no play on the head of the finger grip 11, and is initially positioned, as represented in FIGS. 6 and 7, so that the pins 60 of the branches 58, 59 are accommodated in the catches 43 of the locking tabs 25, 26.

This locking member 51 is thus in a deactivation position in which any relative movement apart of the locking fingers 22, 23 is prevented, and as a result any unlocking of said locking fingers.

As previously, the first actuation of the plunger 5 for the purpose of emptying the syringe does not cause any change of the position of the locking member.

Whereas, as represented in FIG. 11, during drawing up of the mixture inside the syringe, the intermediate collar 32 comes up against the cross-piece 57 and brings about a deformation of the locking part 55 and in particular of the branches 58, 59 which bring about a positioning of the pins beyond the locking tabs 25, 26, for which said branches no longer prevent a relative movement apart of the locking fingers 22, 23.

By the addition of a simple locking member, the invention therefore leads to transforming a single-use safety injection device into a safety injection device allowing, without needing any specific action from the user, a first actuation of the plunger, then the injection itself accompanied by security protection.

1. Safety device for injecting a medicinal product such as a lyophilized product, comprising a syringe body (8) with a receptacle (1) which has an injection needle (3) and equipped with a plunger (5) actuated by a plunger rod (6) equipped with a push-button (7), and a protection device comprising:
a protective casing (19) adapted so that said protective casing and the syringe body (8) are able to slide relatively one in relation to the other between an injection position in which the injection needle (3) extends at least partially into the extension of the protective casing (19), and a protection position after use in which the injection needle (3) is completely accommodated in the protective casing (19),
and at least one locking member (22, 23) which can have a locked state for holding the syringe body (8) and the protective casing (19) in their injection position, and switch to an unlocked state which can allow, at the end of the movement of the plunger (5) inside the receptacle (1), the relative sliding of the syringe body (8) and the protective casing (19) to their protection position after use,
said injection device being characterised in that the protection device comprises:
a shoulder (32) placed in an intermediate position of the length of the plunger rod (6),
a locking member (33; 51) attached on one of the members, syringe body (8) or protective casing (19) comprising:
blocking devices (40, 41; 58; 59) of the locking members (22, 23) that can be displaced between a natural initial position, called the deactivation position, of blocking each locking member (22, 23) in its locked state, and a position, called the activation position, which allows each locking member (22, 23) to be switched to its unlocked state,
and actuating members (44; 57) connected to the blocking devices (40, 41; 58; 59), positioned on the trajectory of the intermediate shoulder (32) of the plunger rod (6), and adapted to undergo a change of state bringing about a displacement of said blocking devices from their deactivation position to the activation position, when a force is exerted on said actuating members by the intermediate shoulder (32), during a displacement of the plunger rod (6) resulting from a tension exerted on the push-button (7).

2. Injection device according to claim 1 characterised in that:
the locking member (33) is attached on one of the members, syringe body (8) or protective casing (19), with an ability of movement according to a direction parallel to the longitudinal axis of the protection device, between two positions defining the respective positions of deactivation and activation of the blocking devices (40, 41) of the locking members (22, 23),
the actuating members comprise at least one lever (44) articulated on the locking member (33) around a rotation axis (45) and comprising, extending on both sides of said rotation axis;
a contact area (46) situated on the trajectory of the intermediate shoulder (32) of the plunger rod (6),
and at least one buttressing part (47, 48) which can bring about a movement of the locking member (33) resulting in a displacement of the blocking devices (40, 41) from their deactivation position to their activation position, during a rotation of the lever (44) caused by the displacement of the plunger rod (6) resulting from a tension exerted on the push-button (7).

3. Injection device according to claim 1 characterised in that:
the locking member (51) is attached in a fixed position on one of the members, syringe body (8) or protective casing (19),
the actuating members comprise at least one locking part (55) interdependent on the locking member (51), comprising a single holder
a contact area (57) situated on the trajectory of the intermediate shoulder (32) of the plunger rod (6),
at least one blocking device (58, 59) of one locking member (22, 23),
and a deformable area (56) of connection between the contact area (57) and each blocking device (58; 59) which can deform and bring about a displacement of each blocking device (58; 59) from its deactivation position to its activation position, when a force is exerted on the contact area (57) by the intermediary shoulder (32) during a displacement of the plunger rod (6) resulting from a tension exerted on the push-button (7).

4. Injection device according to claim 1 characterised in that the locking member (33; 51) is attached on the syringe body (8).

5. Injection device according to claim 1 characterised in that the blocking devices (40, 41; 58; 59) of the locking member (33; 51) and the locking members (22, 23) for holding the syringe body (8) and the protective casing (19) in their injection position, comprise additional removable assembly members (42, 43; 60, 43) of the catch/pin type.

6. Injection device according to claim 1 in which the locking members (22, 23), for holding the syringe body (8) and the protective casing (19) in their injection position, consist of flexible strips, characterised in that the blocking devices (40, 41; 58; 59) of said locking members, are adapted to prevent, in their deactivation position, the deformation of the latter by bending.

7. Injection device according to claim 2 characterised in that the locking member (33; 51) is attached on the syringe body (8).

8. Injection device according to claim 3 characterised in that the locking member (33; 51) is attached on the syringe body (8).

9. Injection device according to claim 2 characterised in that the blocking devices (40, 41; 58; 59) of the locking member (33; 51) and the locking members (22, 23) for holding the syringe body (8) and the protective casing (19) in their injection position, comprise additional removable assembly members (42, 43; 60, 43) of the catch/pin type.

10. Injection device according to claim 3 characterised in that the blocking devices (40, 41; 58; 59) of the locking member (33; 51) and the locking members (22, 23) for holding the syringe body (8) and the protective casing (19) in their injection position, comprise additional removable assembly members (42, 43; 60, 43) of the catch/pin type.

11. Injection device according to claim 4 characterised in that the blocking devices (40, 41; 58; 59) of the locking member (33; 51) and the locking members (22, 23) for holding the syringe body (8) and the protective casing (19) in their injection position, comprise additional removable assembly members (42, 43; 60, 43) of the catch/pin type.

12. Injection device according to claim 2 in which the locking members (22, 23), for holding the syringe body (8) and the protective casing (19) in their injection position, consist of flexible strips, characterised in that the blocking devices (40, 41; 58; 59) of said locking members, are adapted to prevent, in their deactivation position, the deformation of the latter by bending.

13. Injection device according to claim 3 in which the locking members (22, 23), for holding the syringe body (8) and the protective casing (19) in their injection position, consist of flexible strips, characterised in that the blocking devices (40, 41; 58; 59) of said locking members, are adapted to prevent, in their deactivation position, the deformation of the latter by bending.

14. Injection device according to claim 4 in which the locking members (22, 23), for holding the syringe body (8) and the protective casing (19) in their injection position, consist of flexible strips, characterised in that the blocking devices (40, 41; 58; 59) of said locking members, are adapted to prevent, in their deactivation position, the deformation of the latter by bending.

15. Injection device according to claim 5 in which the locking members (22, 23), for holding the syringe body (8) and the protective casing (19) in their injection position, consist of flexible strips, characterised in that the blocking devices (40, 41; 58; 59) of said locking members, are adapted to prevent, in their deactivation position, the deformation of the latter by bending.
